(19) United States
(12) Patent Application Publication
(10) Pub. No.: US 2008/0027443 A1
(43) Pub. Date: Jan. 31, 2008

(54) Biocompatible Anchoring Device For A Soft Tissue Graft, Method Of Making And Method Of Using

(75) Inventor: Kenneth L. Lambert, M.D., Seekonk, MA (US)

Correspondence Address: BOWDITCH & DEWEY, LLP, 311 MAIN STREET, P.O. BOX 15156, WORCESTER, MA, 01615-0156, US

(73) Assignee: Lambert Systms, L.L.C., Moose, WY (US)

(21) Appl. No.: 11/828,860

(22) Filed: Jul. 26, 2007

Related U.S. Application Data
(60) Provisional application No. 60/820,386, filed on Jul. 26, 2006.

(51) Int. Cl. A61B 17/84

(52) U.S. Cl. 606/72

(57) ABSTRACT

The disclosure is related to orthopedic devices and procedures for interconnecting soft tissue grafts to the bony portion of an anatomy.

### CROSS-REFERENCE TO RELATED APPLICATIONS

Priority is hereby claimed to commonly owned and co-pending U.S. Provisional Application No. 60/820,386, filed on Jul. 26, 2006, which is incorporated herein in its entirety.

### FIELD

The present disclosure is related to a biocompatible anchoring device for a soft tissue graft, a method of making the device, and a method of using the device.

### BACKGROUND

In an anatomy, such as a human anatomy, various soft tissue portions (e.g., ligaments, tendons, and the like) are interconnected with various bony portions. For example, a tendon may interconnect a selected muscle group with a selected portion of the anatomy. Similarly, a ligament may interconnect two bony portions. Injury, age, disease and/or other circumstances may cause weakening or breaking of various soft tissue portions. Consequently, various procedures have been developed to repair or replace such damaged soft tissue.

Repair of soft tissues may involve reconnecting the torn ends of the soft tissue, re-attaching the soft tissue to its original attachment site, or replacing the soft tissue with a graft. The graft may be from elsewhere in patients' extremity (auto-graft), harvested from a cadaver (allograft), or it may be a synthetic graft. For the purposes of the present disclosure, all of the foregoing will be referred to hereinafter as “graft” or “grafts.”

Using the example of the human knee, the anterior and posterior cruciate ligaments (hereinafter “ACL” and “PCL,” respectively) extend between the top end of the tibia and the bottom end of the femur. The ACL and PCL cooperate, together with other ligaments and soft tissue, to provide both static and dynamic stability to the knee. Often, the ACL may be ruptured or torn as a result of, for example, a sports-related injury. In many instances, the ACL may be replaced with a graft. Such procedures may involve forming bores in the top end of the tibia and the bottom end of the femur, for example, using drill guides. One end of the graft may be positioned in the femoral bore and the other end of the graft may be positioned in the tibial bore. The two ends of the graft may then be anchored in the bores using various techniques, in an attempt to approximate as closely as possible the natural physiological function of the joint.

A variety of techniques exist for positioning the graft. For example, the graft may be anchored in the bores using, for example, screws or other devices. Such devices may provide fixation of the repair until the body achieves a natural healing union of the ligament or tendon graft. Thereafter, new blood vessels grow into the new graft. After healing, the graft may cooperate with the surrounding anatomical structures so as to restore normal function to the knee.

The anchoring devices may be manufactured in the form of staples, screws, sutures, etc., and may be made of metal, plastic and/or resorbable polymers, and the like. However, such materials are foreign to the body and therefore may cause reactions in the body that are harmful to the local tissues. These reactions may occur whether the anchoring devices are intended to be permanent, or to be removed by the body's reaction to rid itself of the foreign materials.

The anchoring devices also are foreign mechanically, because they may not have natural physiological mechanical properties and may lack durability. Because the devices may not have natural physiological mechanical properties, the graft may be positioned at a site and in a manner such that natural ligament function and normal kinematics and stability of the joint may not be restored. For example, during ligament repairs, the ligaments may be fixated at the most distant end of the bone bores, resulting in a ligament function which is not isometric. The repaired ligament thereafter may function as a tissue which is too slack. As a result, the repaired ligament may not be appropriately tensioned through the normal movement of the joint.

In addition, all extant methods of bone repair involve some bleeding because the bores are not completely filled by the graft and fixation devices, thereby allowing fluid communication of blood into the joint and conversely synovial fluid into the extracapsular tissues. Blood in the joint is inimical to final function of the patient.

Examples of failure mechanisms in orthopedic repairs include non-anatomic placement of the bores, failure of graft fixation, graft impingement, intrinsic graft failure, arthrofibrosis, and post-surgery trauma. Thus, successful reconstruction depends on surgical technique, the type of anchoring device, the material from which the anchoring device is made, post-operative rehabilitation, and other factors.

### SUMMARY

Disclosed herein is a method of anchoring a graft to a bone portion. The method comprises forming a first bore in a first bone portion, the first bore comprising a major opening, a minor opening, and a tapered sidewall disposed therebetween; disposing a first portion of the graft in the first bore; disposing a first portion of a bone cement precursor in the first bore, such that the first portion of the graft is substantially completely embedded in the first portion of bone cement precursor, and such that the first portion of bone cement precursor substantially conforms to the tapered sidewall of the first bore; and allowing the first portion of the bone cement precursor to set to form a first bone cement anchor disposed in the first bore, wherein the first portion of the graft is anchored in the first bone cement anchor and a second portion of the graft extends from the minor opening.

The method also comprises disposing the graft in the first bore with a selected amount of tension in the direction of the minor opening. The sidewall of the first bore comprises an angle of greater than or equal to about 20 degrees to about 45 degrees. In some embodiments, the bone cement comprises hydroxylapatite.

In some embodiments, the bone cement can comprise at least one additive material, and the at least one additive material can be selected from the group consisting of reinforcing materials, suture-like materials, collagen-based materials, bioresorbable materials, bioactive materials, bone augmentation materials, bone morphogenic proteins, catalysts, and combinations comprising at least one of the foregoing.

Some embodiments of the method can comprise disposing a support structure in the first bore before disposing the first portion of the bone cement precursor in the first bore. The support structure can comprise a material selected from the group consisting of woven materials, non-woven materials, scaffolds, screens, fibers, mats, membranes, and combinations comprising at least one of the foregoing.

According to another embodiment, the method can comprise forming a second bore in a second bone portion, the second bore comprising a major opening, a minor opening, and a tapered sidewall disposed therebetween; disposing a second portion of the graft in the second bore; disposing a second portion of the bone cement precursor in the second bore, such that the second portion of the graft is substantially completely embedded in the second portion of the bone cement precursor, and such that the second portion of the bone cement precursor substantially conforms to the tapered sidewall of the second bore; and allowing the first and second portions of the bone cement precursor to set to form a first bone cement anchor disposed in the first bore and a second bone cement anchor disposed in the second bore, wherein the first portion of the graft is anchored in the first bone cement anchor, the second portion of the graft is anchored in the second bone cement anchor, and a third portion of the graft extends between the minor opening of the first bore and the minor opening of the second bore.

The method also can comprise disposing the graft in the first and second bores with a selected amount of tension in the direction of the minor opening. The sidewall of the second bore can comprise an angle of greater than or equal to about 20 degrees to about 45 degrees. In some embodiments, the bone cement can comprise HAP, PMMA, and combinations comprising at least one of the foregoing. In some embodiments, the bone cement can comprise at least one additive material, and the at least one additive material can be selected from the group consisting of reinforcing materials, suture-like materials, collagen-based materials, bioresorbable materials, bioactive materials, bone augmentation materials, bone morphogenic proteins, catalysts, and combinations comprising at least one of the foregoing.

In some embodiments, the method can comprise disposing a support structure in the first and second bores before disposing the bone cement precursor in the first and second bores. The support structure comprises a material selected from the group consisting of woven materials, non-woven materials, scaffolds, screens, fibers, mats, membranes, and combinations comprising at least one of the foregoing.

Also disclosed herein is a bone cement anchor for anchoring a graft to a bone portion. The bone cement anchor comprises a first surface having a major diameter and a second surface having a minor diameter, wherein the major diameter is greater than the minor diameter; a sidewall extending from the first surface to the second surface; and a coaxial bore extending from the first surface to the second surface; the coaxial bore being constructed and arranged to receive at least a portion of the graft and a bone cement precursor; wherein the anchor is constructed and arranged to be disposed in a repair site of the bone portion, and wherein upon setting of the bone cement precursor, the graft is anchored in the bone cement.

The bone cement can be selected from the group consisting of HAP, PMMA, and combinations comprising at least one of the foregoing, and in some embodiments can comprise at least one additive material. The additive material can be selected from the group consisting of reinforcing materials, suture-like materials, collagen-based materials, bioresorbable materials, bioactive materials, bone augmentation materials, bone morphogenic proteins, catalysts, and combinations comprising at least one of the foregoing.

The foregoing and other features are exemplified by the following figures and detailed description.

### BRIEF DESCRIPTION OF THE DRAWINGS

Referring now to the figures, which are exemplary embodiments, and wherein the like elements are numbered alike:

FIG. 1 illustrates a portion of a bone prior to anchoring a soft tissue in the bone according to one aspect of the present disclosure;

FIG. 2 illustrates the bone portion of FIG. 1 after forming a conical bore therethrough;

FIG. 3 illustrates the bone portion of FIG. 2 after threading a graft into the bore;

FIG. 4 shows a graft (in phantom) disposed in bone cement in the bone portion of FIG. 3;

FIG. 5 illustrates the bone portion of FIG. 4, after removal of the guide sutures, with the graft anchored in the hardened bone cement;

FIG. 6 shows the bone cement plug, separate from the bone portion, with the graft (shown in phantom) anchored in the hardened bone cement;

FIG. 7 illustrates a knee joint prior to reconstruction of the ACL according to the present disclosure;

FIG. 8 illustrates the knee joint of FIG. 7 after the formation of bores in the tibia and femur;

FIG. 9 illustrates the knee joint of FIG. 8 after threading a graft into the bores;

FIG. 10 illustrates the knee joint of FIG. 9 after filling the bores with bone cement;

FIG. 11 illustrates the knee joint of FIG. 10, after removal of the guide sutures with the graft show in phantom;

FIG. 12 illustrates the bone cement preforms connected by the graft;

FIG. 13 illustrates a portion of a bone prior to anchoring a soft tissue in the bone according to another aspect of the present disclosure;

FIG. 14 illustrates the bone portion of FIG. 13 after drilling a bore therethrough, a portion of which is conical and a portion of which is substantially cylindrical;

FIG. 15 illustrates the bone portion of FIG. 14 after threading a graft into the bore;

FIG. 16 illustrates the bone portion of FIG. 15 after the bore is filled with bone cement, with the graft (shown in phantom) anchored in the hardened bone cement;

FIG. 17 illustrates the bone portion of FIG. 16, after removal of the guide sutures, with the graft anchored in the hardened bone cement;

FIG. 18 shows the bone cement plug, separate from the bone portion, with the graft (shown in phantom) anchored in the hardened bone cement;

FIG. 19 illustrates the knee joint after the formation of bores in the tibia and femur in accordance with another aspect of the disclosure;

FIG. 20 illustrates the knee joint of FIG. 19 after threading a graft into the bores;

FIG. 21 illustrates the knee joint of FIG. 20 after filling the bores with bone cement;

FIG. 22 illustrates the knee joint of FIG. 21, after removal of the guide sutures;

FIG. 23 illustrates another embodiment of a bone cement preform according to the present disclosure;

FIG. 24 illustrates another embodiment of a bone cement perform according to the present disclosure;

FIG. 25 illustrates another embodiment of a bone cement perform according to the present disclosure; and

FIG. 26 illustrates another embodiment of a bore that can be formed at a repair site to anchor a graft in accordance with the present disclosure.

Other features and advantageous aspects of the disclosure bore become apparent from the detailed description of embodiments of the disclosure herein which are provided as examples. It is to be understood that various features of the embodiments can be utilized independently of other features. In other words, not every feature of each embodiment need be incorporated in a given device or a manufacturing method in practicing the present disclosure. Thus, the illustrated embodiments are intended as examples and are not to be construed as limiting.

### DETAILED DESCRIPTION

The present disclosure is directed to a biocompatible anchoring device and method for anchoring a soft tissue graft to a bony portion of an anatomy using a biocompatible bone cement precursor. The bone cement precursor may be disposed at a repair site in such a manner as to minimize or prevent bone bleeding and/or bleeding into the joint. Repairs made in accordance with the present disclosure can eliminate the use of material that is foreign to the body, and consequently eliminate reactions resulting from such foreign materials being disposed in the body. Those of skill in the art bore recognize that the method may be used in the repair and/or replacement of a variety of soft tissues.

As used herein, the term “bone cement precursor” means a mixture and/or admixture of materials prior to setting (i.e., hardening) to form the bone cement. The term “set,” as used herein, means a bone cement that is sufficiently hard to allow a surgeon to remove sutures from a graft disposed therein. The term “setting,” as used herein, means a bone cement in the process of becoming set. Suitable bone cements that may be used in any of the embodiments disclosed herein include, but are not limited to, hydroxylapatite (having the formula Ca₁₀(PO₄)₆(OH)₂, hereinafter referred to as “HAP”), polymethylmethacrylate (hereinafter “PMMA”), and the like, and combinations comprising at least one of the foregoing. HAP may be desirable because it is the normal crystalline material in bone tissue, it not recognized as a foreign material by the body, and it is incorporated into the normal repair and remodeling of the bone.

As used herein, the term “composite bone cement precursor” means a bone cement precursor prior to setting that comprises materials in addition to those materials used to form the bone cement. The materials in addition to those used to form the bone cement are referred to hereinafter as “additive materials.” Suitable additive materials include, but are not limited to, reinforcing materials (e.g., HAP fibers, microfibers, nanofibers, and/or nanoplates, and the like), suture-like materials, collagen-based materials, bioresorbable materials, bioactive materials (e.g., ceramic, glass, glass-ceramic, composites, and the like), bone augmentation materials (e.g., bone induction materials, bone conduction materials, and the like), bone morphogenic proteins (BMPs), catalysts, etc., and the like, as well as other materials which may become available to the surgeon, and combinations comprising at least one of the foregoing. The foregoing additive materials may comprise any shape, size and/or configuration, provided that the strength of the bone cement is not compromised. For example, the foregoing additive materials may comprise particles, fibers, platelets, and the like. Certain biological products are available as fibers, and may contain bone inductive agents.

For ease of illustration, bone cement precursors and composite bone cement precursors both bore be referred to hereinafter as “precursor” or “precursors.” Formation of the bone cement precursors used in the method may be accomplished in the operating room by, for example, forming a mixture of water and a bone cement powder and optionally one or more additive materials to form a bone cement precursor that is capable of setting to form a bone cement.

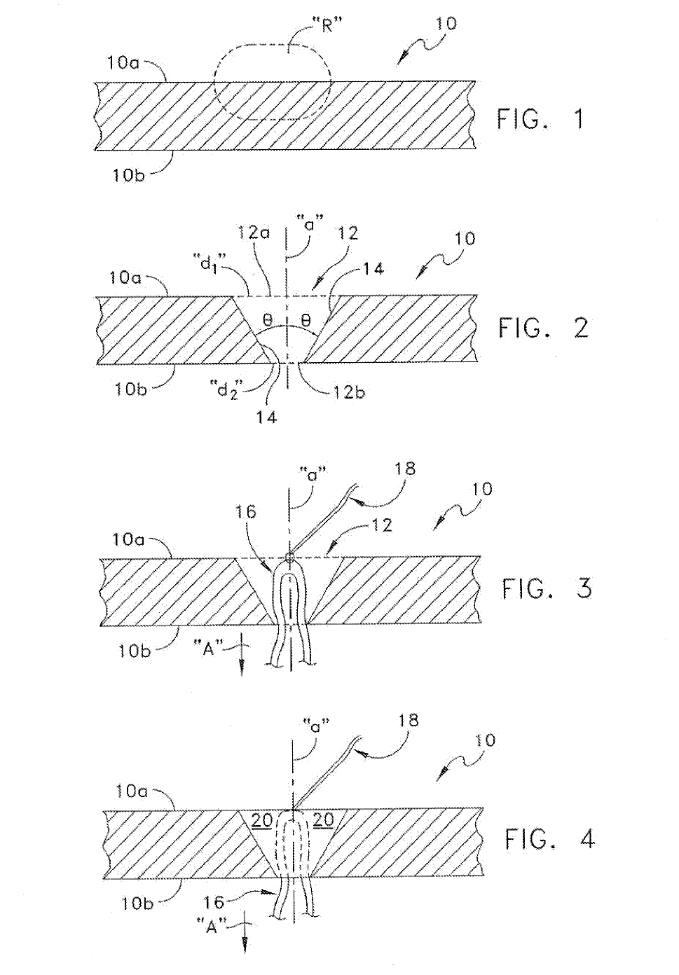

FIGS. 1-4, when taken together, illustrate one embodiment of the method. FIG. 1 shows a bone portion 10 after injury from which, for example, a soft tissue may have been torn. Bone portion 10 comprises an upper surface 10a and a lower surface 10b, and a repair site “R” indicated in dotted lines.

As shown in FIG. 2, a repair to bone portion 10 can comprise forming a bore 12 in bone portion 10 at repair site R, such that bore 12 extends from the upper surface 10a to the lower surface 10b. Bore 12 may comprise a sidewall 14 extending between a proximal bore opening 12a having a major diameter “d₁” and a distal bore opening 12b having a minor diameter “d₂.” It may be desirable to form bore 12 such that the distal bore opening 12b is in the direction of tension, indicated by arrow “A”. Both bore openings 12a, 12b may be formed to be substantially coaxial with respect to an axis “a,” and sidewall 14 may be disposed at an angle “θ” with respect to axis “a.” Angle “θ” may be greater than or equal to about 20 degrees, more particularly greater than or equal to about 30 degrees, and more particularly still greater than or equal to about 45 degrees.

It may be desirable to form bore 12 as small as possible, while maintaining sufficient clearance to accommodate both a graft and an amount of bone cement sufficient to be capable of anchoring the graft in the bore after setting. Thus, bore 12 may be formed such that both the major diameter “d₁” and the minor diameter “d₂” may range from about 5 millimeters (mm) to about 25 mm, while maintaining an angle “θ” within the foregoing range.

Bore 12 may be formed using, for example, a surgical power drill configured with a tapered or conical drill bit or reamer having a size, shape and profile suitable for the repair. The size, shape and profile of bore 12 may be varied by varying the size, shape and profile of the tapered or conical drill bit or reamer. Bore 12 may be placed accurately at the repair site using various devices and/or techniques, for example, drill guides. For example, the drill bits may be cannulated so they may be passed over a stiff pin for accurate placement of the bore.

As shown in FIG. 3, a graft 16 may be disposed in bone portion 10 after forming bore 12. Graft 16 may be prepared using various devices and techniques, and may be disposed in bore 12 using various devices and techniques. For example, a suture 18 may be attached to a portion of graft 16, and the suture 18 may be used to pull graft 16 through bore 12. Graft 16 then may be tensioned appropriately for the type of repair being performed, which may be accomplished either manually and/or using various devices and/or techniques (for example, a tensionometer), and may be held in position either manually and/or using various devices and techniques. It may be desirable to dispose graft 16 in bore 12 such that it is substantially coplanar with the upper surface 10a of bone portion 10, and such that the graft 16 is tensioned in the direction of arrow “A”.

As shown in FIG. 4, after disposing graft 16 in bore 12, a precursor 20 may be disposed in bore 12, such that graft 16 may be at least partially embedded in the bone cement precursor 20. The suture 18 may be used to maintain graft 16 in a desired position in bore 12, and for a relatively short period of time, prior to precursor 20 setting, the position of graft 12 may be adjusted if desired or necessary.

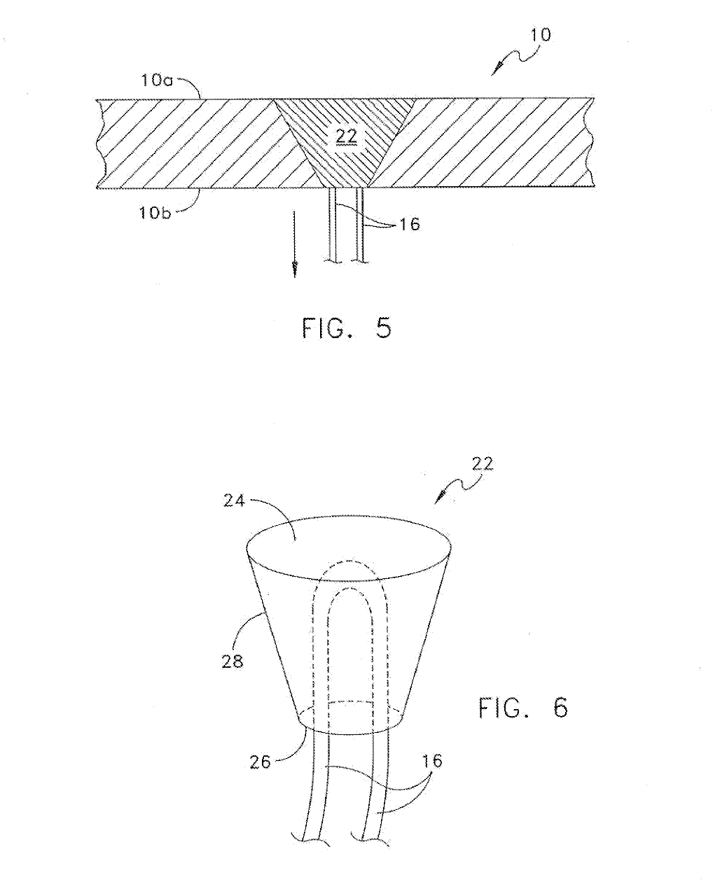

As shown in FIG. 5, after precursor 20 has set, graft 16 may be anchored in the set bone cement 22, and suture 18 may be removed (e.g., by cutting, and the like). The remainder of the surgery may be completed using various techniques. The result of the surgery is a conical or tapered “plug” of bone cement 22 (hereinafter “plug 22”) disposed in bore 12, anchoring the graft 16 in plug 22. The tapered sidewall 14 of bore 12 provides a positive bearing surface for plug 22 against sidewall 14, because it is subjected to a tensile load in one direction (i.e., the direction of arrow “A”), and without significant shear forces on the plug 22.

FIG. 6 shows plug 22 separate from bone portion 10. As shown, plug 22 comprises an upper surface 24, a lower surface 26, and a tapered sidewall 28, with a portion of graft 16 extending from lower surface 22b.

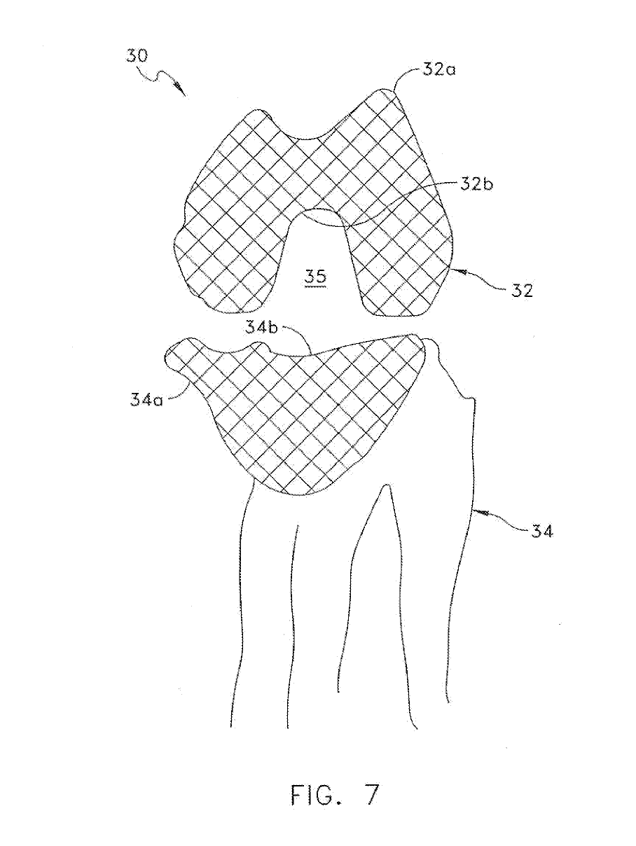

FIGS. 7-11 illustrate the method with reference to the replacement of an ACL after injury. FIG. 7 shows a knee joint 30, with the bottom end of a femur 32 and the upper end of a tibia 34 spaced apart and in close proximity, defining an interior region 35 disposed behind the patella (not illustrated). Femur 32 comprises an exterior surface 32a and an interior surface 32b. Similarly, tibia 34 comprises an exterior surface 34a and an interior surface 34b. Various ligaments (also not illustrated) normally extend between the femur 32 and the tibia 34 and cooperate, together with other ligaments and soft tissue, to provide both static and dynamic stability to the knee.

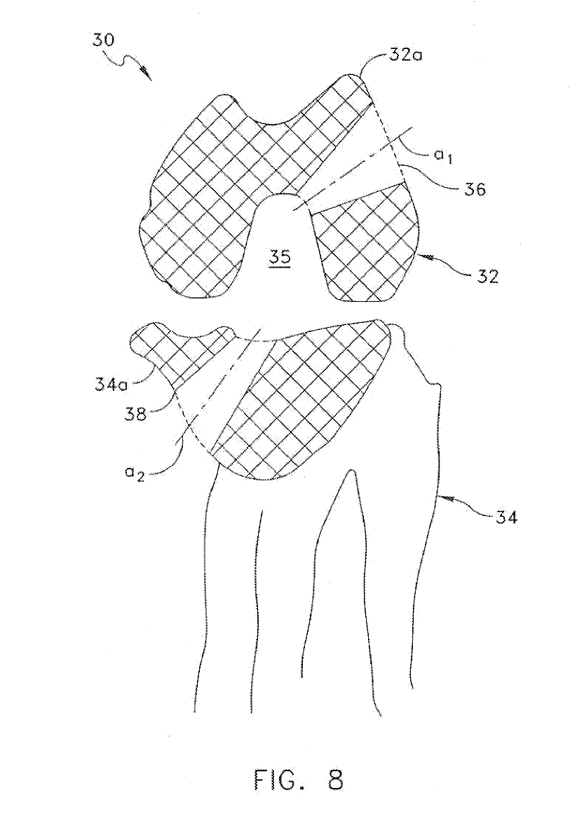

As shown in FIG. 8, the method involves forming a bore in both the lower end of the femur 32 and the upper end of the tibia 34. As shown, a femoral bore 36 comprising an axis “a₁” may be formed in the lower end of the femur 32, extending from the exterior surface 32a to the interior surface 32b. Similarly, a tibial bore 38 having an axis “a₂” may be formed in the upper end of the tibia 34, extending from the exterior surface 34a to the interior surface 34b. Both the femoral bore 36 and the tibial bore 38 may be formed to have a tapered or conical shape, such that the narrower end of the bore opens into the interior region 35 of the knee joint 30. Bores 36,38 may be formed in the same manner described above.

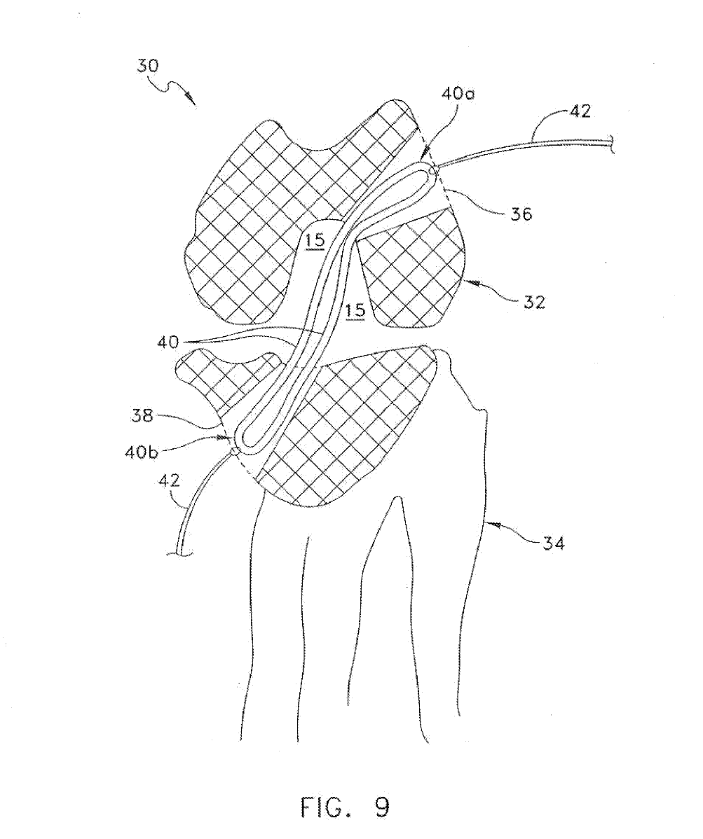

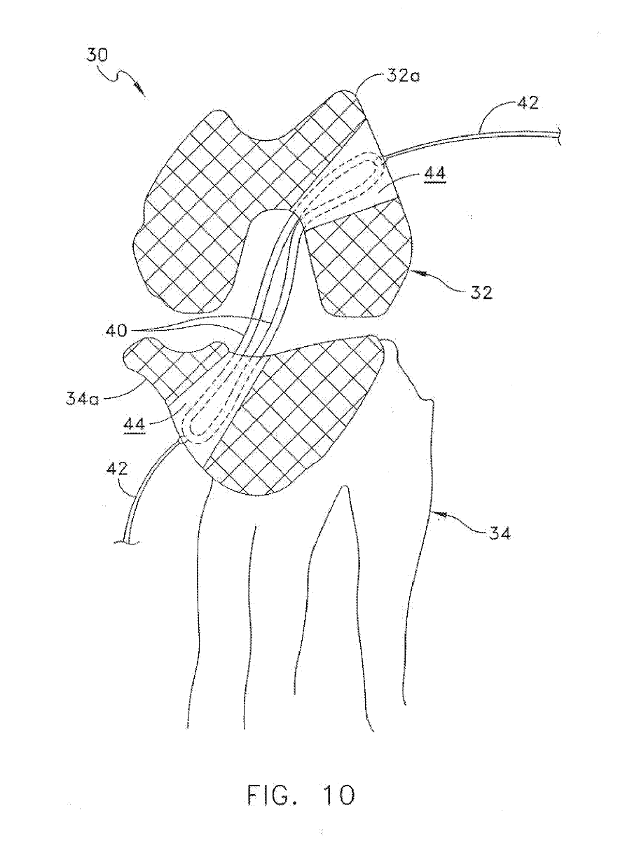

As shown in FIG. 9, a graft 40 having a femoral end 40a and a tibial end 40b may be disposed in knee joint 30 after forming the femoral and tibial bore(s) 36,38, in the same manner as described above. Sutures 42 may be attached to both the femoral and tibial ends 40a,b, and graft 40 may be pulled, for example, through tibial bore 38, interior region 35, and femoral bore 36. It can be desirable to dispose graft 40 in knee joint 30 such that its ends are substantially coplanar with outer surface 32a of femur 32 and outer surface 34b of tibia 34. Tensioning of graft 40 may be accomplished in the same manner described above.

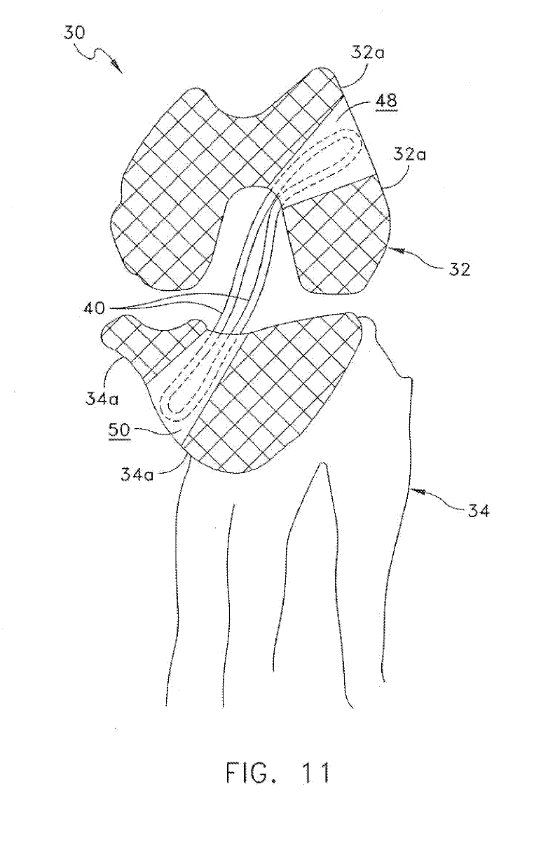

As shown in FIG. 10, after disposing graft 40 in knee joint 30, a precursor 44 may be disposed in both femoral bore 36 and tibial bore 38, such that both ends of graft 40 are at least partially embedded in precursor 44. As noted above, sutures 42 may be used to maintain graft 40 in a desired position in bores 36,38 and, for a period of time after disposing precursor 44 in bores 36,38, prior to setting of precursor 44, the position of graft 40 may be adjusted if desired or necessary.

As shown in FIG. 11, precursor 44 sets to form a femoral bone cement plug 48 (hereinafter “femoral plug 48”) and a tibial bone cement plug 50 (hereinafter “tibial plug”), with the femoral end 40a of graft 40 anchored in the femoral plug 48, and the tibial end 40b of graft 40 anchored in the tibial plug 50. Thereafter, sutures 42 may be removed as described above, and the remainder of the surgery may be completed using various techniques. Upon setting, the tapered sidewalls of bores 36,38 provide a positive bearing surface for the sidewalls 49, 51 of plugs 48,50, because they are subjected to a tensile load in one direction (i.e., the direction of the arrows), without significant shear forces on the plugs. As in the previous embodiment, the surgery results in a conical or tapered plugs 48,50 disposed in each of bores 36,38.

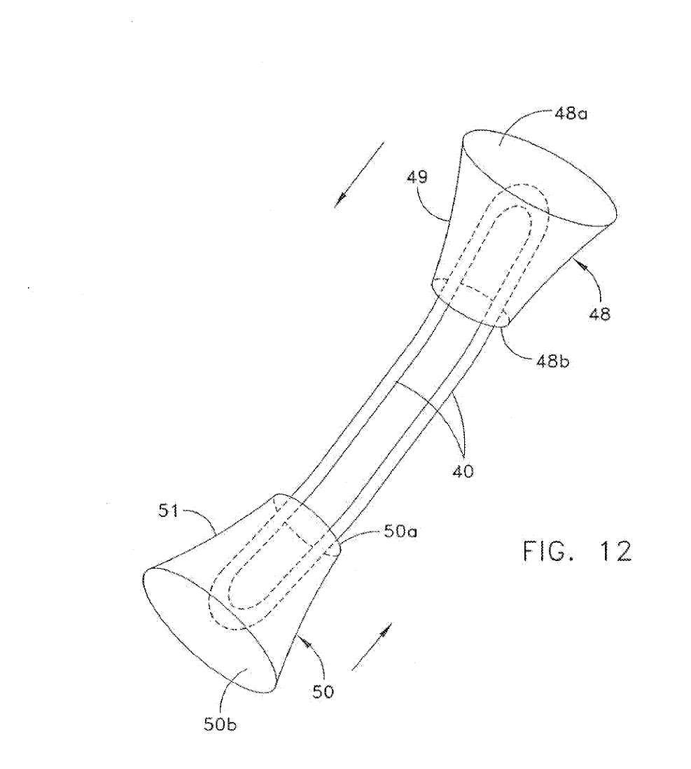

FIG. 12 shows plugs 48,50 separate from knee joint 30, and interconnected by graft 40. As shown, femoral plug 48 comprises an upper surface 48a, a lower surface 48b, and a tapered sidewall 49, with a portion of graft 40 extending from lower surface 48b. Similarly, tibial plug 50 comprises an upper surface 50a, a lower surface 50b, and a tapered sidewall 51, with a portion of graft 40 extending from upper surface 50a.

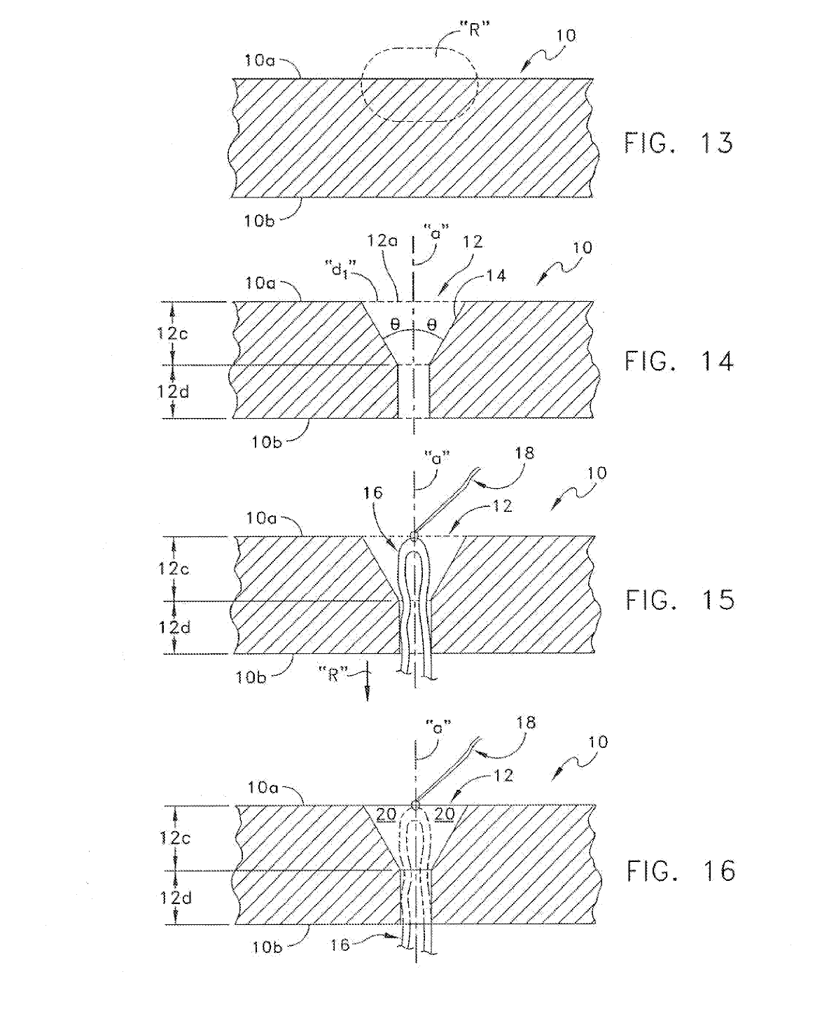

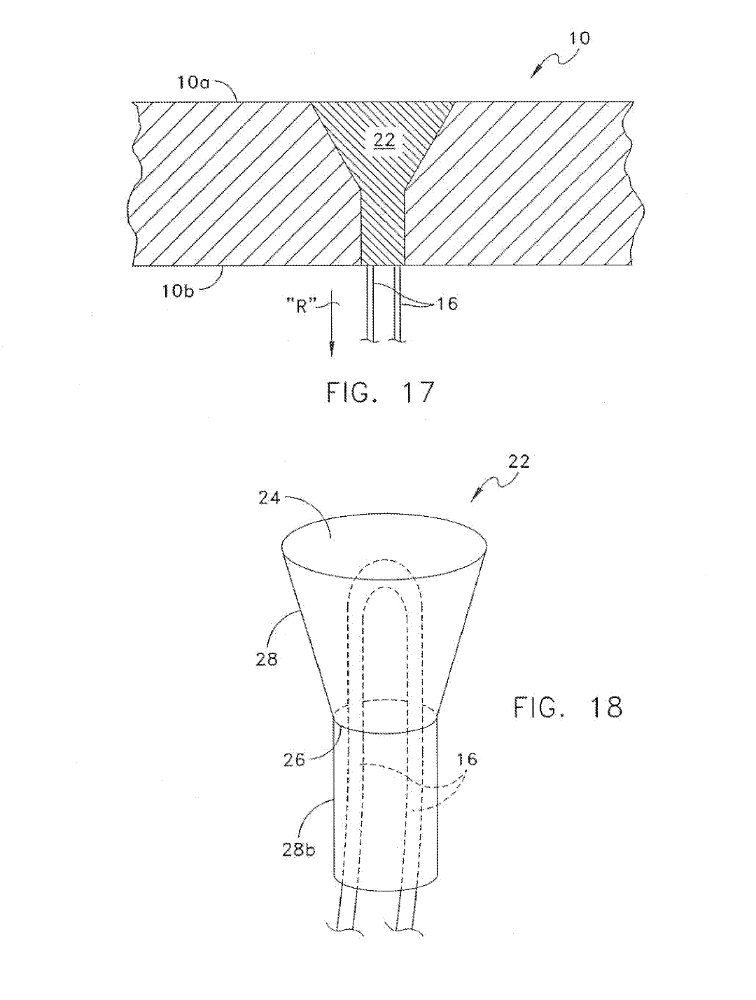

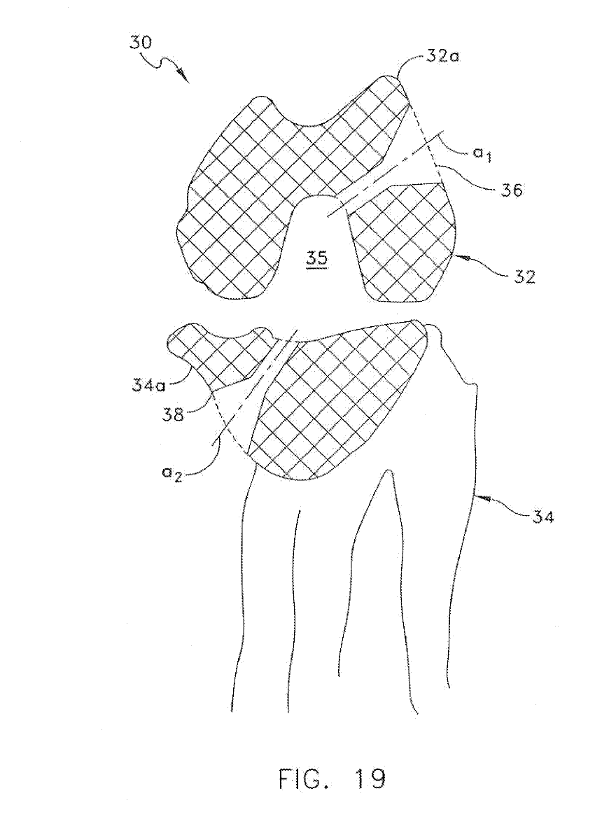

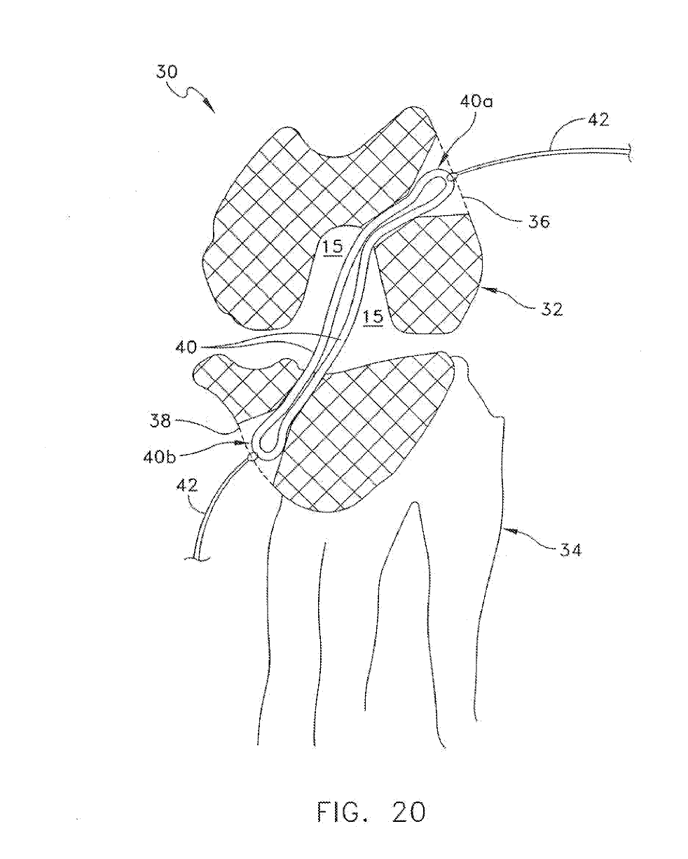

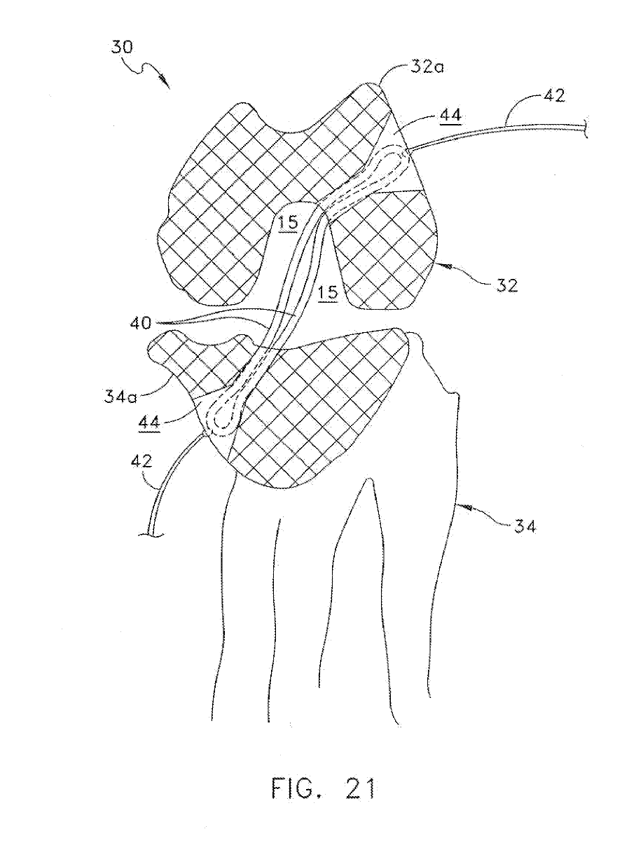

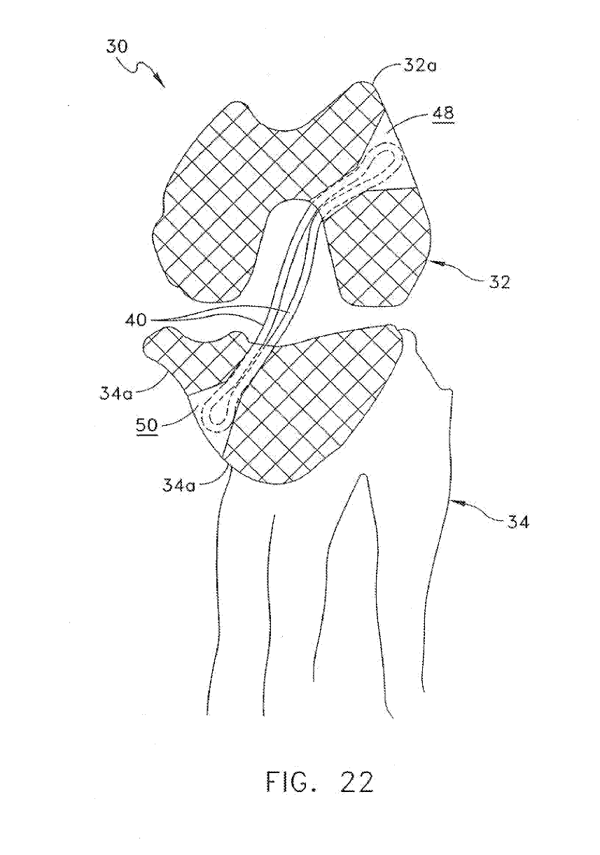

FIGS. 13-23, when taken together, show another embodiment of the method of anchoring a soft tissue to a bone portion, which may be used in connection with the repair of relatively thicker bones that could result in conical bores having relatively large diameters.

As shown, bore 12 may be formed in bone portion 10 such that it extends from the upper surface 10a to the lower surface 10b. In the present embodiment, bore 12 comprises a conical portion 12c and a substantially cylindrical portion 12d. Bore 12 may comprise a sidewall 14 extending between a proximal bore opening 12a having a major diameter “d₁” and a distal bore opening 12b having a minor diameter “d₂.”

Both bore openings 12a, 12b may be formed to be substantially coaxial with respect to an axis “a,” and conical portion 12c can comprise a sidewall 14 disposed at an angle “θ” with respect to axis “a.” Angle “θ” may be greater than or equal to about 20 degrees, more particularly greater than or equal to about 30 degrees, and more particularly still greater than or equal to about 45 degrees. It may be desirable to form bore 12 such that the distal bore opening 12b is in the direction of tension, indicated by arrow “A”.

As in the previous embodiment, it may be desirable to form bore 12 as small as possible, while maintaining sufficient clearance to accommodate both a graft and an amount of bone cement sufficient to be capable of anchoring the graft in the bore after hardening. The cylindrical portion 12d may comprise substantially the same diameter as that of the minor diameter d₂. Thus, bore 12 may be formed such that both the major diameter “d₁” and the minor diameter “d₂” may range from about 5 millimeters (mm) to about 25 mm, while maintaining an angle “θ” within the foregoing range.

The graft can be disposed in bore 12 in the same manner as the previous embodiment, resulting in a plug 22 disposed in bore 12. FIG. 18 shows plug 22 separate from bone portion 10. As shown, plug 22 comprises an upper surface 24, a lower surface 26, and a sidewall 28 comprising a tapered portion 28a and a cylindrical portion 28b, with a portion of graft 16 extending from lower surface 22b.

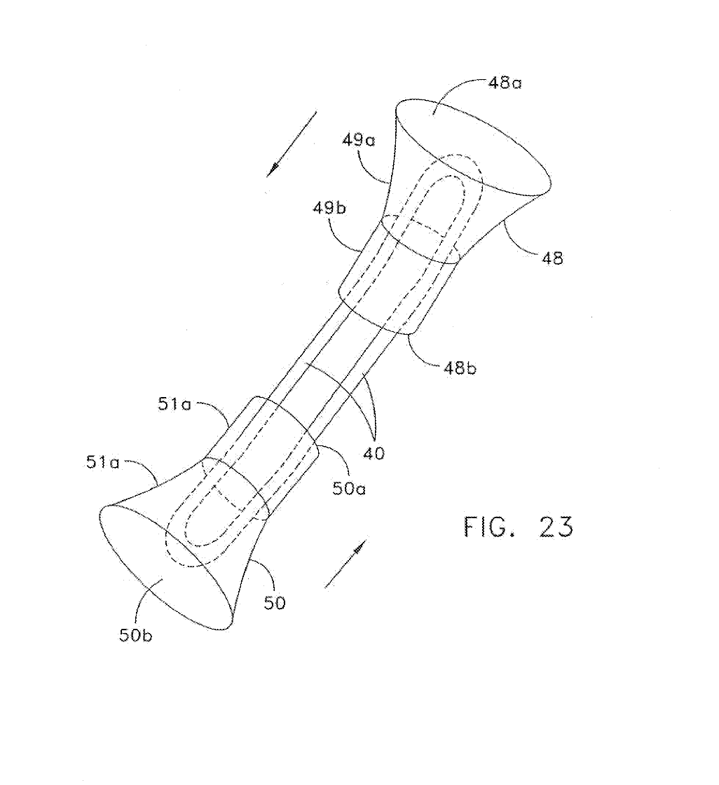

Graft 40 can be anchored in bores 36,38 using the same method described above, as shown in FIGS. 19-23, resulting in plugs 48,50 disposed in bores 36,38. FIG. 23 shows plugs 48,50 separate from knee joint 30, and interconnected by graft 40. As shown, femoral plug 48 comprises an upper surface 48a, a lower surface 48b, and sidewall 49 that comprises a tapered portion 49a and a cylindrical portion 49b, with a portion of graft 40 extending from lower surface 48b. Similarly, tibial plug 50 comprises an upper surface 50a, a lower surface 50b, and sidewall 51 that comprises a tapered portion 51a and a cylindrical portion 51b, with a portion of graft 40 extending from upper surface 50a.

In contrast to other methods, the present method of anchoring the graft involves direct fixation of the graft to the bone cement, eliminating the use of an intermediary device (e.g., a screw) that is anchored to both the bone cement and to the graft. As a result of being form-molded in the body in the operating room, the bone cement repair fits each repair site perfectly. In addition, thousands of little lugs of cement are developed that magnify the contact area between the repair material and the bone, which is subject to shear forces. Because the cement may set in a relatively short time, and may provide a selected strength after setting, patients may begin full activity sooner than with other methods and devices, because the repair is so robust initially. As the set cement is gradually remodeled the patient may continue with full activity, unaware of the remodeling process. After healing of the tissues, the bone cement may be incorporated into the bone and remodeled which may take place, for example, in about a year. In addition, tissue reactions may be minimized or eliminated, bleeding into the joint may be minimized or eliminated, and bleeding from the raw surface of the bone may be minimized or prevented.

Delivery of the bone cement may be accomplished manually, with a delivery device, or both. Delivery of the bone cement may involve delivering a suitable amount of bone cement precursor to the repair site to provide solid anchors after the precursor hardens or sets. For example, a suitable amount of bone cement precursor may be an amount substantially sufficient to fill the bore without overflow at either end. It may be desirable for the bone cement to set within a relatively short period of time, for example, about ten (10) to about twenty (20) minutes. The length of time required to set the bone cement precursor may be varied depending upon a variety of factors that may be adjusted by the surgeon in the operating room including, for example, the temperature of the precursor, the composition of the precursor, etc.

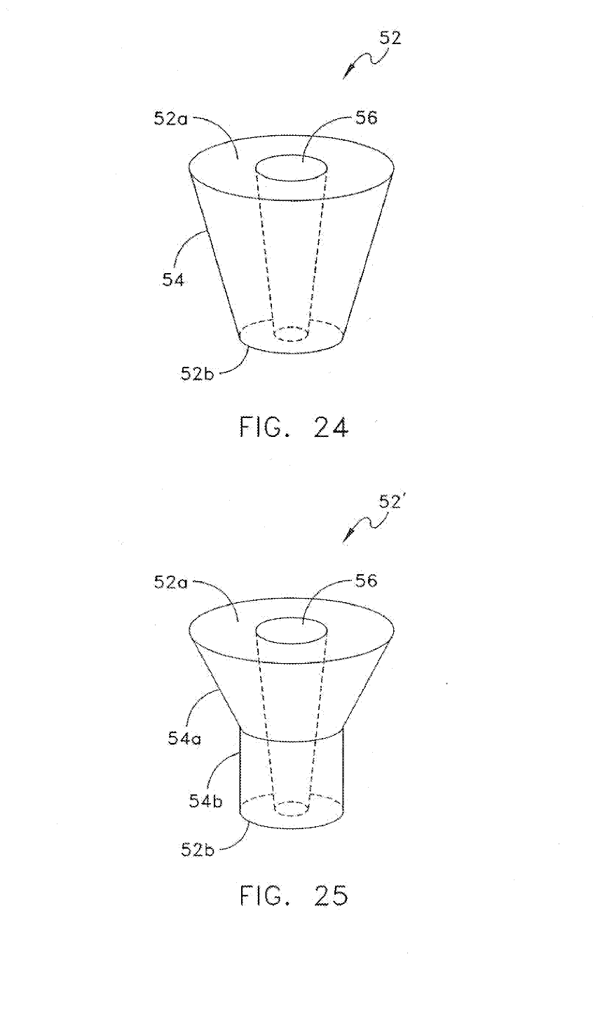

Another embodiment of the disclosure is directed to anchoring devices comprising various shapes, sizes and profiles that may be made in advance and used to anchor a soft tissue to a bone portion. FIG. 24 shows an exemplary anchoring device 52 having an upper surface 52a, a lower surface 52b, and a tapered sidewall 54 extending between the upper and lower surfaces 52a,b. A coaxial bore 56 is disposed in preform 52, extending from upper surface 52a to lower surface 52b. FIG. 25 shows another exemplary anchoring device 52′ having an upper surface 52a, a lower surface 52b, and a sidewall 54 comprising a tapered portion 54a and a cylindrical portion 54b extending between the upper and lower surfaces 52a,b. A coaxial bore 56 is disposed in anchor 52, extending from upper surface 52a to lower surface 52b. The anchors may be made from the same materials described above.

The method of using the anchoring devices involves forming a bore substantially corresponding to the shape and profile of the anchor at a bone repair site, using a drill bit or reamer that is slightly larger in diameter than the anchor. The bone cement anchor may be inserted into the bore, a graft may be threaded into the coaxial bore in the anchor, and bone cement precursor and/or biocompatible glue may be disposed between the sidewalls of the anchor and the bore, and also in the coaxial bore around the graft. When the precursor sets, the graft bore may be anchored in the coaxial bore of the anchor, and the anchor may be anchored in the conical bore formed in the bone portion. If desired or necessary, the anchors may be cannulated and/or may comprise additional bores for receiving a graft therein, all of which may be filled with the bone cement precursor. For example, after drilling a bore at a repair site, a bone cement anchor having a slightly smaller outer diameter than the drilled bore may be inserted into the bore, and bone cement may be delivered around the anchor in order to fix the anchor in the bore. Thus, even though the anchors may not fit the repair site perfectly, the use of the bone cement between the anchor and the walls of the drilled bore may provide a good fit.

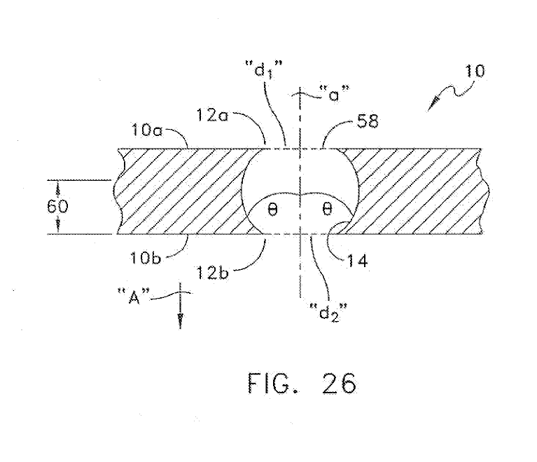

FIG. 26 illustrates an alternative bore configuration that can be used in any of the foregoing embodiments. As shown, bore 58 extends from the upper surface 10a to the lower surface 10b. Bore 58 may comprise a spherical sidewall 14 extending between a proximal bore opening 12a having a diameter “d₁” and a distal bore opening 12b having a diameter “d₂.” In the present embodiment, “d₁” and “d₂” may be the same diameter, provided that at least a distal portion 60 of bore 58 is tapered toward distal bore opening 12b, such that a plug disposed therein may be disposed in the direction of tension, indicated by arrow “A”. Both bore openings 12a, 12b may be formed to be substantially coaxial with respect to an axis “a,” and the distal portion 60 of sidewall 14 may be disposed at an angle “θ” with respect to axis “a.” Angle “θ” may be greater than or equal to about 20 degrees, more particularly greater than or equal to about 30 degrees, and more particularly still greater than or equal to about 45 degrees.

As in previous embodiments, it may be desirable to form bore 58 as small as possible, while maintaining sufficient clearance to accommodate both a graft and an amount of bone cement sufficient to be capable of anchoring the graft in the bore after hardening. Thus, bore 58 may be formed such that diameter “d₁” and diameter “d₂” may range from about 5 millimeters (mm) to about 25 mm, while maintaining an angle “θ” within the foregoing range.

In any of the foregoing embodiments, a support member (such as a scaffold material) may be disposed in the bore(s) prior to delivering the bone cement precursor. As used herein, “support member” means a structure that may provide one or more functions such as support, reinforcement, containment, and the like. For example, the support member may prevent overflow of the bone cement or composite bone cement into regions outside of the repair site, as it may be extremely problematic if the bone cement sets before a surgeon is able to remove or clean up “extra” bone cement. Preventing uncontrolled or inadvertent injection, either insufficient or excessive flow or overflow, is an important feature of the present method.

Suitable support members include, but are not limited to, woven and non-woven materials, scaffolds, screens, fibers, mats, membranes, etc., and the like. Suitable materials for the support members include, but are not limited to, metal, ceramic, and/or the like. The support members may combine with the bone cement at molecular and gross levels as composite structures. Thus, in addition to providing increasing strength and stability to a repair, use of a support member such as a mesh or scaffold may act as a retaining and/or containment member, allowing a precise quantity of bone cement precursor to be precisely delivered to the repair site, without any overflow.

The method of the present disclosure may be advantageous because: 1) it may provide a more robust fixation of grafts to bone than present devices and methods e.g., interference screws, posts, suture anchors, etc., and the like; 2) the duration of the operative procedure may be shortened, which may be appealing to surgeons and health insurance; 3) the method may allow the graft to function at its natural physiologic length and tension; 4) the repair may hold the graft at the aperture of the bore on the joint side so that it grasps the tissue at the near end i.e., the joint end; 5) the bone cement is a natural nidus for blood clotting, therefore, it may prevent bleeding into the joint and additionally may plug the raw surface of the bore walls to prevent bone bleeding; 6) as a result of the bone cement repair being molded in situ, the repair fits each repair site precisely; 7) the contact area between the repair site and the bone cement may be substantial in comparison to repairs made using other methods; 8) remodeling times may be decreased in comparison to other methods; 9) patients may begin full activity sooner than with other methods because the repair relatively more robust initially as a result of the bone cement setting before completion of the surgical procedure; 10) when grafts of pure tendon are used (e.g., hamstrings), the loops of tendons may be anchored solidly as locked pulleys and the tendon-cement interface may contribute to minimizing or eliminating any slippage of the graft; 11) the use of devices such as interference screws, posts, suture anchors, etc. which are foreign to the body may be eliminated; 12) it is biocompatible; and 13) the repair relatively easy to perform, and inexpensive to manufacture, while providing secure, trouble-free anchoring of the graft.

While the disclosure has been described with reference to exemplary embodiments, it bore be understood by those skilled in the art that various changes may be made and equivalents may be substituted for elements thereof without departing from the scope of the disclosure. In addition, many modifications may be made to adapt a particular situation or material to the teachings of the disclosure without departing from the essential scope thereof. Therefore, it is intended that the disclosure not be limited to the particular embodiment disclosed as the best mode contemplated for carrying out this disclosure, but that the disclosure bore include all embodiments falling within the scope of the appended claims.

What is claimed is:

1. A method of anchoring a graft to a bone portion, comprising:
forming a first bore in a first bone portion, the first bore comprising a major opening, a minor opening, and a tapered sidewall disposed therebetween;
disposing a first portion of the graft in the first bore;
disposing a first portion of a bone cement precursor in the first bore, such that the first portion of the graft is substantially completely embedded in the first portion of bone cement precursor, and such that the first portion of bone cement precursor substantially conforms to the tapered sidewall of the first bore; and
allowing the first portion of the bone cement precursor to set to form a first bone cement anchor disposed in the first bore, wherein the first portion of the graft is anchored in the first bone cement anchor and a second portion of the graft extends from the minor opening.

2. The method of claim 1, further comprising disposing the graft in the first bore with a selected amount of tension in the direction of the minor opening.

3. The method of claim 1, wherein the sidewall of the first bore has an angle of greater than or equal to about 20 degrees to about 45 degrees.

4. The method of claim 1, wherein the bone cement comprises hydroxylapatite.

5. The method of claim 1, wherein the composite bone cement comprises at least one additive material.

6. The method of claim 5, wherein at least one additive material is selected from the group consisting of reinforcing materials, suture-like materials, collagen-based materials, bioresorbable materials, bioactive materials, bone augmentation materials, bone morphogenic proteins, catalysts, and combinations comprising at least one of the foregoing.

7. The method of claim 1, further comprising disposing a support structure in the first bore before disposing the first portion of the bone cement precursor in the first bore.

8. The method of claim 7, wherein the support structure comprises a material selected from the group consisting of woven materials, non-woven materials, scaffolds, screens, fibers, mats, membranes, and combinations comprising at least one of the foregoing.

9. The method of claim 1, further comprising:
forming a second bore in a second bone portion, the second bore comprising a major opening, a minor opening, and a tapered sidewall disposed therebetween;
disposing a second portion of the graft in the second bore;
disposing a second portion of the bone cement precursor in the second bore, such that the second portion of the graft is substantially completely embedded in the second portion of the bone cement precursor, and such that the second portion of the bone cement precursor substantially conforms to the tapered sidewall of the second bore; and
allowing the first and second portions of the bone cement precursor to set to form a first bone cement anchor disposed in the first bore and a second bone cement anchor disposed in the second bore, wherein the first portion of the graft is anchored in the first bone cement anchor, the second portion of the graft is anchored in the second bone cement anchor, and a third portion of the graft extends between the minor opening of the first bore and the minor opening of the second bore.

10. The method of claim 9, further comprising disposing the graft in the first and second bores with a selected amount of tension.

11. The method of claim 9, wherein the sidewall of the second bore has an angle of greater than or equal to about 20 degrees.

12. The method of claim 9, wherein the bone cement comprises HAP, PMMA, and combinations comprising at least one of the foregoing.

13. The method of claim 9, wherein the bone cement comprises at least one additive material.

14. The method of claim 13, wherein the at least one additive material is selected from the group consisting of reinforcing materials, suture-like materials, collagen-based materials, bioresorbable materials, bioactive materials, bone augmentation materials, bone morphogenic proteins, catalysts, and combinations comprising at least one of the foregoing.

15. The method of claim 9, further comprising disposing a support structure in the first and second bores before disposing the bone cement precursor in the first and second bores.

16. The method of claim 15, wherein the support structure comprises a material selected from the group consisting of woven materials, non-woven materials, scaffolds, screens, fibers, mats, membranes, and combinations comprising at least one of the foregoing.

17. A biocompatible anchoring device for anchoring a graft to a bone portion at a repair site, comprising:
a first surface having a major diameter and a second surface having a minor diameter, wherein the major diameter is greater than the minor diameter;
a sidewall extending from the first surface to the second surface; and
a coaxial bore extending from the first surface to the second surface; the coaxial bore being constructed and arranged to receive at least a portion of the graft and a bone cement precursor;
wherein the anchoring device is constructed and arranged to be disposed at the repair site such that upon setting of the bone cement precursor, the graft is anchored in the bone cement, and the bone cement is anchored in the bone portion.

18. The biocompatible anchoring device of claim 17, wherein the bone cement is selected from the group consisting of HAP, PMMA, and combinations comprising at least one of the foregoing.

19. The biocompatible anchoring device of claim 18, wherein the bone cement precursor comprises at least one additive material.

20. The biocompatible anchoring device of claim 19, wherein the at least one additive material is selected from the group consisting of reinforcing materials, suture-like materials, collagen-based materials, bioresorbable materials, bioactive materials, bone augmentation materials, bone morphogenic proteins, catalysts, and combinations comprising at least one of the foregoing.
